(19) United States
(12) Patent Application Publication
(10) Pub. No.: US 2005/0239763 A1
(43) Pub. Date: Oct. 27, 2005

(54) Non-GMO metal amino acid chelates and non-GMO metal amino acid chelate-containing compositions

(75) Inventors: Max R. Motyka, St. Clair Shores, MI (US); Rick Harnish, Miami, FL (US); Stephen D. Ashmead, Clinton, UT (US); H. DeWayne Ashmead, Fruit Heights, UT (US)

Correspondence Address: THORPE NORTH & WESTERN, LLP., 8180 SOUTH 700 EAST, SUITE 200, P.O. BOX 1219, SANDY, UT, 84070, US

(73) Assignee: Albion International, Inc.

(21) Appl. No.: 10/829,468

(22) Filed: Apr. 21, 2004

(51) Int. Cl. C07F 15/02; A61K 31/555; A61K 33/24

(52) U.S. Cl. 514/184; 514/503

(57) ABSTRACT

Non-GMO metal amino acid chelate compositions, non-GMO formulations containing non-GMO metal amino acid chelates, methods of preparing non-GMO metal amino acid chelates, and methods of administering non-GMO metal amino acid chelates are provided. Specifically, the present invention provides a non-GMO metal amino acid chelate composition, comprising amino acid chelates having a naturally occurring amino acid to metal molar ratio of from about 1:1 to 4:1, wherein both the amino acid and the source of the metal used to form the amino acid chelate are non-GMO.

### FIELD OF THE INVENTION

The present invention is drawn to non-GMO metal amino acid chelates and non-GMO formulations containing amino acid chelates.

### BACKGROUND OF THE INVENTION

Amino acid chelates are generally produced by the reaction between α-amino acids and metal ions having a valence of two or more to form a ring structure. In such a reaction, the positive electrical charge of the metal ion can be neutralized by the electrons available through the carboxylate or free amino groups of the α-amino acid.

Traditionally, the term “chelate” has been loosely defined as a combination of a polyvalent metallic ion bonded to one or more ligands to form a heterocyclic ring structure. Under this definition, chelate formation through neutralization of the positive charge(s) of the metal ion may be through the formation of ionic, covalent, or coordinate covalent bonding. An alternative and more modern definition of the term “chelate” requires that the polyvalent metal ion be bonded to the ligand solely by coordinate covalent bonds forming a heterocyclic ring. In either case, both are definitions that describe a metal ion and a ligand forming a heterocyclic ring.

Chelation can be confirmed and differentiated from mixtures of components by infrared spectra through comparison of the stretching of bonds or shifting of absorption caused by bond formation. As applied in the field of mineral nutrition, there are certain “chelated” products that are commercially utilized. One product is referred to as an “amino acid chelate.” When properly formed, an amino acid chelate is a stable product having one or more five-membered rings formed by a reaction between the amino acid and the metal. The American Association of Feed Control Officials (AAFCO) has also issued a definition for amino acid chelates. It is officially defined as the product resulting from the reaction of a metal ion from a soluble metal salt with amino acids having a mole ratio of one mole of metal to one to three (preferably two) moles of amino acids to form coordinate covalent bonds. The products are identified by the specific metal forming the chelate, e.g., iron amino acid chelate, copper amino acid chelate, etc.

In further detail with respect to amino acid chelates, the carboxyloxygen and the α-amino group of the amino acid each bond with the metal ion. Such a five-membered ring is defined by the metal atom, the carboxyloxygen, the carbonyl carbon, the α-carbon, and the α-amino nitrogen. The actual structure will depend upon the ligand to metal mole ratio and whether the carboxyloxygen forms a coordinate covalent bond or a more ionic bond with the metal ion. Generally, the amino acid to metal molar ratio is at least 1:1 and is preferably 2:1 or 3:1. However, in certain instances, the ratio can be 4:1. Most typically, an amino acid chelate with a divalent metal can be represented at a ligand to metal molar ratio of 2:1 according to Formula 1 as follows: 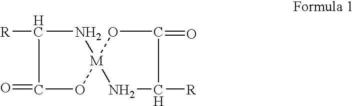
In the above formula, the dashed lines represent coordinate covalent bonds, covalent bonds, or ionic bonds. Further, when R is H, the amino acid is glycine, which is the simplest of the α-amino acids. However, R could be representative of any other side chain that, when taken in combination with the rest of the amino acid structure(s), results in any of the other twenty or so naturally occurring amino acids that are typically derived from proteins. All of the amino acids have the same configuration for the positioning of the carboxyloxygen and the α-amino nitrogen with respect to the metal ion. In other words, the chelate ring is defined by the same atoms in each instance, even though the R side chain group may vary.

The reason a metal atom can accept bonds over and above the oxidation state of the metal is due to the nature of chelation. For example, at the α-amino group of an amino acid, the nitrogen contributes to both of the electrons used in the bonding. These electrons fill available spaces in the d-orbitals forming a coordinate covalent bond. Thus, a metal ion with a normal valency of +2 can be bonded by four bonds when fully chelated. In this state, the chelate is completely satisfied by the bonding electrons and the charge on the metal atom (as well as on the overall molecule) can be zero. As stated previously, it is possible that the metal ion can be bonded to the carboxyloxygen by either coordinate covalent bonds or more ionic bonds.

The structure, chemistry, bioavailability, and various applications of amino acid chelates are well documented in the literature, e.g. Ashmead et al., Chelated Mineral Nutrition, (1982), Chas. C. Thomas Publishers, Springfield, Ill.; Ashmead et al., Intestinal Absorption of Metal Ions, (1985), Chas. C. Thomas Publishers, Springfield, Ill.; Ashmead et al., Foliar Feeding of Plants with Amino Acid Chelates, (1986), Noyes Publications, Park Ridge, N.J.; U.S. Pat. Nos. 4,020,158; 4,167,564; 4,216,143; 4,216,144; 4,599,152; 4,725,427; 4,774,089; 4,830,716; 4,863,898; 5,292,538; 5,292,729; 5,516,925; 5,596,016; 5,882,685; 6,159,530; 6,166,071; 6,207,204; 6,294,207; 6,458,981, 6,518,240, 6,614,553; each of which is incorporated herein by reference.

One advantage of amino acid chelates in the field of mineral nutrition is attributed to the fact that these chelates are readily absorbed from the gut and into mucosal cells by means of active transport. In other words, the minerals can be absorbed along with the amino acids as a single unit utilizing the amino acids as carrier molecules. Therefore, the problems associated with the competition of ions for intestinal absorption sites and the suppression of specific nutritive mineral elements by others can be avoided.

Issues surrounding the use of genetically modified organisms, or GMOs, have become more prevalent in recent years due to the great advances in genetic engineering. However, with the advance of technology related to GMOs and GMO derivatives (products produced from GMOs, such as proteins), there are a large number of people who view this type of genetic manipulation as undesirable. Critics of GMO technology believed that continuing to carry out genetic transfer after genetic transfer, sooner or later, unforeseen consequences may result. Possible unforeseen consequences that might occur include problems ranging from minor allergic reactions to environmental disasters based upon unanticipated changes in aggressiveness of disease organisms. Another segment of society sees the entire process of moving genes among species as unjustifiable and immoral, as such genetic engineering is unnatural.

Regardless of the merits of the concerns, there is growing need in the food and plant industries to provide an alternative to GMOs and GMO derivatives, if for no other reason than to avoid unnecessary regulation. For example, when genetic modification involves plant foods, the U.S. Department of Agriculture can become involved in regulation. Further, when dealing with pest management, the U.S. Environmental Protection Agency has a regulatory interest.

Due at least in part to health and environmental concerns, as well as due to the regulatory concerns, it would be an advancement in the art to provide non-GMO metal amino acid chelates and non-GMO formulations that contain amino acid chelates.

### SUMMARY OF THE INVENTION

It has been recognized that the preparation of non-GMO metal amino acid chelates and formulations containing such chelates would be beneficial. In accordance with this, a non-GMO metal amino acid chelate composition can comprise a metal amino acid chelate including a naturally occurring amino acid chelated to a metal. The amino acid to metal molar ratio can be from about 1:1 to 4:1. Additionally, both the amino acid and the source of the metal used to form the amino acid chelate are non-GMO.

In another embodiment, a non-GMO metal amino acid chelate-containing composition can comprise a non-GMO metal amino acid chelate and a non-GMO additive. The non-GMO metal amino acid chelate can include a naturally occurring amino acid chelated to a metal, wherein amino acid to metal molar ratio being from about 1:1 to 4:1. Both the amino acid and the source of the metal used to form the amino acid chelate in this embodiment are non-GMO. Additionally, if any other components are present in the composition, those components are also non-GMO.

In another embodiment, a method of preparing a non-GMO metal amino acid chelate can comprise steps of selecting an amino acid source determined to be non-GMO and selecting a metal source determined to be non-GMO. Another step includes chelating an amino acid of the amino acid source to a metal of the metal source, thereby forming a non-GMO metal amino acid chelate.

In still another embodiment, a method of administering a metal amino acid chelate can comprise formulating a non-GMO metal amino acid chelate and administering the non-GMO metal amino acid chelate to the subject. The step of formulating can be by selecting an amino acid source determined to be non-GMO, selecting a metal source determined to be non-GMO, and chelating an amino acid of the amino acid source to a metal of the metal source, thereby forming the non-GMO metal amino acid chelate.

Additional features and advantages of the invention will be apparent from the following detailed description which illustrates, by way of example, features of the invention.

### DETAILED DESCRIPTION OF THE PREFERRED EMBODIMENT(S)

Before the present invention is disclosed and described, it is to be understood that this invention is not limited to the particular process steps and materials disclosed herein because such process steps and materials may vary somewhat. It is also to be understood that the terminology used herein is used for the purpose of describing particular embodiments only. The terms are not intended to be limiting because the scope of the present invention is intended to be limited only by the appended claims and equivalents thereof.

It is to be noted that, as used in this specification and the appended claims, the singular forms “a,” “an,” and “the” include plural referents unless the context clearly dictates otherwise.

The term “naturally occurring amino acid” or “traditional amino acid” shall mean amino acids that are known to be used for forming the basic constituents of proteins, including alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and combinations thereof. The term “naturally occurring” does not mean that the amino acid used in accordance with embodiments of the present invention is necessarily derived naturally, but that it can occur naturally.

The term “amino acid chelate(s)” is intended to cover both the traditional definitions and the more modern definition of chelate as cited previously. Specifically, with respect to chelates that utilize traditional amino acid ligands, i.e. those used in forming proteins, chelate is meant to include metal ions bonded to amino acid ligands forming heterocyclic rings. Between the carboxyloxygen and the metal, the bond can be covalent or more ionic, but is preferably coordinate covalent. Additionally, at the α-amino group, the bond is typically a covalent or coordinate covalent bond.

When referring to “amino acid chelates” or “metal amino acid chelates” in the plural form, this phraseology does not necessarily infer that two distinct amino acid chelates are present. For example, a particulate batch of a single species of an amino acid chelate can be referred to as “amino acid chelates.” Alternatively, the term “amino acid chelates” can also include multiple types of amino acid chelates in a batch, depending on the context.

The term “nutritionally relevant metal” is meant to include any polyvalent, e.g., divalent or trivalent, metal that can be used as part of a nutritional supplement, drug therapy, food fortificant, topical cosmetic, etc., that is known to be beneficial to animals including humans, and in some instances, plants. Nutritionally relevant metals are also known to be substantially non-toxic when administered in traditional amounts, as is known in the art. Examples of such metals include iron, zinc, copper, manganese, calcium, magnesium, chromium, vanadium, selenium, silicon, molybdenum, tin, nickel, boron, cobalt, gold, silver, and combinations thereof.

The term “GMO” is an acronym for the term “genetically modified organism(s).”

The term “GMO derivative” applies to any substance produced from, but not containing a genetically modified organism.

The term “non-GMO” herein includes compositions that are not GMOs, and also are not derived from GMOs. In other words, non-GMO compositions are not genetically modified of themselves, and are prepared by processes other than those which include the use of genetically modified organisms. Thus, amino acid chelates prepared in accordance with embodiments of the present invention, such as for human, animal, or foliar application, must not include or be produced with the utilization of genetically modified organisms.

Amino acids prepared by “synthetic” methods include chemical preparations that do not involve protein hydrolysis.

Amino acids prepared by “fermentation” methods typically include a bioprocess wherein an engineered or unengineered cell or organism produces the amino acids, usually on a relatively large scale.

To illustrate the concern related to genetically modified organisms, one can consider the genetic modification of crops, many of which can be used to form amino acids and subsequently used to form amino acid chelates. Over the last few years, there has been growing public concern about the impact that genetically modified crops will have on both the environment and public health. Some studies indicate that there are potential problems linked to the use of GMOs. For example, genes from genetically modified crops can be transferred to wild relatives and non-gentically modified crops. Further, crops that are genetically modified to be herbicide-tolerant can threaten the biodiversity in agricultural areas. Insect pests may rapidly develop resistance to genetically modified crops expressing toxins, thus shortening the useful life of certain crops and compromising the effectiveness of existing insecticides. Additionally, it will be difficult to ensure that genetically modified foods will not cause new allergies. From a business perspective, whether or not these and other concerns will be significant in the long run, labeling requirements in conjunction with public perceptions make the use of genetically modified organisms in products financially impractical.

With this in mind, a non-GMO metal amino acid chelate composition can comprise a metal amino acid chelate including a naturally occurring amino acid chelated to a metal. The amino acid to metal molar ratio can be from about 1:1 to 4:1. Additionally, both the amino acid and the source of the metal used to form the amino acid chelate are non-GMO.

In another embodiment, a non-GMO metal amino acid chelate-containing composition can comprise a non-GMO metal amino acid chelate and a non-GMO additive. The non-GMO metal amino acid chelate can include a naturally occurring amino acid chelated to a metal, wherein amino acid to metal molar ratio being from about 1:1 to 4:1. Both the amino acid and the source of the metal used to form the amino acid chelate in this embodiment are non-GMO. Additionally, if any other components are present in the composition, those components are also non-GMO.

A method of preparing a non-GMO metal amino acid chelate is also disclosed, and can comprise steps of selecting an amino acid source determined to be non-GMO and selecting a metal source determined to be non-GMO. Another step includes chelating an amino acid of the amino acid source to a metal of the metal source, thereby forming a non-GMO metal amino acid chelate.

In another embodiment, a method of administering a metal amino acid chelate can comprise formulating a non-GMO metal amino acid chelate and administering the non-GMO metal amino acid chelate to the subject. The step of formulating can be by selecting an amino acid source determined to be non-GMO, selecting a metal source determined to be non-GMO, and chelating an amino acid of the amino acid source to a metal of the metal source, thereby forming the non-GMO metal amino acid chelate.

With respect to the method of preparing and the method of administering, during the selecting steps, determining whether the source material used to form the chelate is non-GMO may result in the consideration of multiple sources before selecting. For example, during the step of selecting the amino acid source, if a first amino acid source is a GMO, additional amino acid sources can be evaluated until a non-GMO amino acid source is ascertained. Additionally, during the step of selecting the metal source, if a first metal source is a GMO, additional metal sources are evaluated until a non-GMO metal source is ascertained.

Determining whether a composition or its source is non-GMO indicates that some type of evaluative step be performed. For example, in determining whether an amino acid, including its source, as well as a metal source is non-GMO, an evaluation step can include steps such as reviewing literature or interviewing manufacturers associated with a product obtained from a third party, preparing the compositions or sources in-house to ensure that all components are and preparations are non-GMO, and/or conducting an assay to verify that a composition is truly non-GMO. A typical assay or test that can be conducted to verify that a composition is non-GMO includes polymerase chain reaction (PCR) analysis, among other known tests. Companies that will conduct GMO studies include Genescan, operating in the U.S., Europe, Brazil, and Hong Kong; Genetic ID of Fairfield, Iowa; and Strategic Diagnostics Inc. of Newark, Del.

Non-GMO Metal Amino Acid Chelates

In accordance with embodiments of the present invention, amino acid chelates that are non-GMO can be prepared by reacting a non-GMO amino acid source with a non-GMO metal source. The non-GMO amino acid source can be a free amino acid or a salt of an amino acid, provide the amino acid or salt of the amino acid is not derived from a genetically modified organism. Likewise, similar considerations can occur with respect to the metal source. Steps of preparing or selecting non-GMO amino acid sources as well as preparing or selecting non-GMO metal sources can be carried out to achieve a desired result.

Exemplary metals that can be used include iron, zinc, copper, calcium, magnesium, and/or manganese, which are common nutritional minerals used when supplementing the mineral balance of subjects, including humans. Further, trace metals, such as chromium, vanadium, selenium, silicon, molybdenum, tin, nickel, boron, cobalt, gold, and/or silver, or the like, can also be used. Regarding the metals that can be prepared or selected for use, metal sources that may be derived from genetically modified organisms can be avoided. For example, biological sources of metal may more likely include genetically modified material. Examples of metals from biological sources that are possible candidates of being derived from genetically modified sources includes heme iron from hemoglobin, magnesium from chlorophyll, calcium from lactose, and magnesium from magnesium stearate. These sources are not precluded from use, provided they are non-GMO sources. Examples of metal sources that typically are not derived from genetically modified material include metal sulfates, metal carbonates, metal oxides, metal hydroxides, elemental metals, and the like.

Examples of amino acid sources that can be non-GMO include those not prepared by protein hydrolysis, those wherein the amino acid source is prepared by protein hydrolysis using a non-GMO protein, amino acids prepared synthetically, and amino acids prepared by fermentation using microorganisms that are not genetically modified. The naturally occurring amino acids that can be used include alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and combinations thereof.

Specific examples of preferred amino acid chelates that can be used include embodiments wherein the amino acid to metal molar ratio is about 2:1, and wherein the metal is ferrous iron and the naturally occurring amino acid is glycine, the metal is copper and the naturally occurring amino acid is glycine, the metal is zinc and the naturally occurring amino acid is glycine, or the metal is manganese and the naturally occurring amino acid is glycine. Alternatively, the amino acid to metal molar ratio can be about 3:1, the metal can be trivalent as with ferric iron or chromium, and the naturally occurring amino acid can be glycine. In yet another embodiment, the amino acid to metal molar ratio can be about 1:1, the metal can be magnesium or calcium, and the naturally occurring amino acid can be glycine.

There are many methods that can be used to ensure that a resulting amino acid chelate composition is non-GMO. For example, synthetic synthesis of amino acids can be used to provide non-GMO amino acids. In one embodiment, the synthesis of α-amino acids can be by reaction of aldehydes with ammonia and hydrogen cyanide, followed by hydrolysis of the resulting α-aminonitriles. Amino acids prepared by this method are available from Dow Chemical and Chattem Chemicals, Inc., among others. Alternatively, amino acids can be prepared by the formation of azlactones by intramolecular condensation of acylglycines in the presence of acetic anhydride. The reaction of azlactones with carbonyl compounds followed by hydrolysis to the unsaturated α-acylamino acid and by reduction yields the amino acid. These synthetic methods of preparation are exemplary only, and are not intended to be limiting.

Fermentation can also be used to prepare amino acids that are non-GMO, provided the microorganism used to prepare the amino acids has not been genetically modified. Amino acid fermentation is a method for producing amino acids using microorganisms to convert nutrients to amino acids. Specifically, raw materials, such as broths or syrups, can be added to microorganism culture media, and the microorganisms are allowed to produce the amino acids. For example, L-amino acids can be accumulated in a fermentation broth, from which the amino acids are isolated and purified. A common amino acid producer includes mutants of coryneform bacteria represented by the genera Corynebacterium and Brevibacterium. Mutants of various types, such as are obtained by mutation and selection (auxotrophic mutants, regulatory mutants, auxotrophic-regulatory mutants) can be used to form the non-GMO amino acids. However, the use of amino acid producers obtained by methods of gene manipulation is outside the scope of the present invention. In another example, some producers are able to synthesize amino acids from such carbon sources as sugar, ethanol, or methanol under optimal conditions of aeration. These conditions can be very different for the individual amino acids. Amino acids overproduction is influenced by the mechanisms of metabolic regulations (on the level of both activity and expression) and amino acid secretion (as diffusion and carrier-mediated membrane transport).

Other amino acid preparative process are described in part or in whole the following articles: Determination of Amino Acids in Cell Cultures and Fermentation Broths, Dionex Application Note 150, pp 1-15; Production of Amino Acids by Analog-Resistant Mutants of Cyanobacterium Spirulina platenis, Riccardi, G. et al., Journal of Bacteriology, pp. 102-107 (September 1981); Cattle Nutrition—Mycotoxins and Intoxications, various authors, Abstracts—XXII World Buiatrics Congress 2002, Hannover, Germany (Aug. 18-23, 2002—Abstract Nos. 1-364, 2-689, 3-229, 4-788, 5-755, 6-157, 7-825, 7-757, 9-226, 10-393, 11-645, 12-904, 13-802); Lysine and other amino acids for feed: production and contribution to protein utilization in animal feeding, Toride, Y. et al.; and Acid-neutralizing activity during amino acid fermentation by Porphyromonas gingivalis, Prevotell intermedia and Fusobacterium nucleatum, Takahashi, N. et al., Oral Microbiology Immunology, vol. 18, no. 2,109-113(5) (April 2003), each of which are incorporated herein by reference in their entireties. The methods disclosed in these articles can be used in accordance with embodiments of the present invention to the extent that they do not use genetically modified organisms to produce the amino acids.

Non-GMO Additives

Depending on the amount of a specific mineral to be administered in an amino acid chelate (or combination of minerals to be administered), non-GMO additives are typically formulated within a common composition with the amino acid chelates to provide desired properties that may not be inherently present in the amino acid chelate itself. As one embodiment of the present invention is drawn non-GMO metal amino acid chelate-containing compositions, care should be taken in selecting additives to administer with the amino acid chelates such that the composition as a whole is non-GMO.

Examples of formulation additives that can be admixed or co-administered with the amino acid chelates of the present invention include non-GMO organic acids, non-GMO free amino acids, non-GMO amino acid salts, non-GMO fillers, non-GMO flow control agents, non-GMO lubricants, non-GMO flow agents, non-GMO hydroscopicity minimizing agents, non-GMO pH control agents, non-GMO catalysts, non-GMO vitamins, non-GMO dust control agents, non-GMO binders, non-GMO disintegrating agents, non-GMO flavoring agents, non-GMO taste-reducing agents, non-GMO capsule shells, non-GMO shellacs, non-GMO waxes, non-GMO emulsifiers, non-GMO oils, combinations thereof, and other known additives that can be prepared to be non-GMO. Many of these compositions are inherently non-GMO, but to the extent that the composition can be prepared by the use of a genetically modified organism, this should be avoided.

There are certain additives that can be formulated to be non-GMO, which can be included in amino-acid chelate-containing compositions that provide desired properties to the composition during formulation or to the finished composition. For example, maltodextrins can be added as a filler and a flow agent. Additionally, maltodextrins can help to reduce the hydroscopicity of the composition as a whole. Grain flours, such as rice flour or wheat flour, can also be added as a filler, as well as vegetable flours or powders, such as soy flour. In another embodiment, a filler that can be added is inulin, such as non-GMO low fiber inulin derived from chicary. Fumed silica, stearic acids, and/or talc can also be added as a flow controlling agents. When including a flow control agent or filler, as described above, care should be taken to select or prepare the additive such that it is non-GMO.

In addition to the flow agents and fillers, other compositions that can be added include organic acids. Citric acid, fumaric acid, succinic acid, tartaric acid, malic acid, lactic acid, gluconic acid, ascorbic acid, pantothenic acid, folic acid, lipoic acid, oxalic acid, maleic acid, formic acid, acetic acid, pyruvic acid, adipic acid, and alpha-ketoglutaric acid are each exemplary of such organic acids, though others can also be used. Free amino acids or amino acid salts can also be present in the composition. Additionally, mineral oils for dust control, binders for tableting (carboxymethyl cellulose, ethyl cellulose, glycerol, etc.), flavoring agents or taste-free additives for organoleptic properties, or the like can also be included.

Other classes of formulation additives that can be included with the non-GMO metal amino acid chelates, which of themselves should also be non-GMO, are vitamins, coenzymes, cofactors, herbs or herbal extracts, protein powders, or the like. Non-GMO vitamins that can be used include Vitamin A, the Vitamin B group of vitamins, e.g., folic acid, Vitamin B₁, Vitamin B₂, Vitamin B₃, Vitamin B₅, Vitamin B₆, or Vitamin B₁₂, Vitamin C, Vitamin D, Vitamin E, and the like. Coenzymes can also be used, which are organic compounds that combine with apoenzymes to form active enzymes. Cofactors that can be present include coenzymes and metals that are required for an enzyme to be active, some of which can be provided by the amino acid chelate itself.

In each of the embodiments described herein, the compositions can be in the form of tablets, capsules, powders, crystals, granules, liquids, or the like. Shellacs or waxes can be used as tablet coatings, provided they are non-GMO. Likewise, if using capsules to deliver a composition in accordance with embodiments of the present invention, the encapsulating material should also be non-GMO. For example, the encapsulating material can be of vegetable sterols or gelatin, for example, provided the encapsulating material is non-GMO, e.g., bovine or porcine gelatin can often be desirable for use. Regarding liquids, compositions can also be included in liquid formulations that act to main the solubility of the amino acid chelate and/or other additives that may be present. For example, U.S. Pat. No. 6,716,814, which is incorporated herein by reference in its entirety, describes a method enhancing the solubility of iron amino acid chelates and iron proteinates. Such methods and solubility enhancing compositions can be used, provided the compositions used are non-GMO.

### EXAMPLES

The following examples are illustrative of the preparation of non-GMO metal amino acid chelates and amino acid chelate-containing formulations. As such, the following examples should not be considered as limitations of the present invention, but merely demonstrate the effectiveness of the methods and compositions described herein.

### Example 1

To about 700 ml of deionized water containing 50 g citric acid is added 225 g of a synthetically produced glycine to form a clear solution. The synthetic production method for preparing the glycine is by reacting aldehydes with ammonia and hydrogen cyanide, followed by hydrolysis of the resulting α-aminonitriles. To this solution of citric acid and glycine is slowly added 55.8 g of elemental iron. The solution is heated at about 50° C. for 48 hours, or until substantially all the iron is observed to go into solution. The product is cooled, filtered, and spray dried yielding an iron triglycine amino acid chelate. All of the compositional components used in the preparation should be determined to be non-GMO.

### Example 2

A solution is prepared including 10.1 parts by weight of fermentation-produced glycine dissolved in 82.2 parts by weight water containing 1.0 part by weight sodium carbonate. To this solution is added 4.4 parts by weight zinc oxide. The molar ratio of glycine to zinc is 2:1. The reaction mixture is allowed to stand for about 14 hours and turned an opalescent color. After standing, the mixture is heated to about 70° C. and is spray dried to obtain a zinc bisglycinate amino acid chelate powder having a melting point of about 209° C. which turned red upon melting. The zinc content of the chelate is about 20 wt %. The dried product has a moisture content of about 7 wt %, and when reconstituted in water, has a pH of about 8.0. All of the compositional components used in the preparation should be determined to be non-GMO.

### Example 3

A copper carbonate solution is prepared by adding 6.1 parts by weight of non-GMO cupric carbonate to 80.9 parts by weight water. This solution is allowed to stand without agitation for about two hours. To this solution is added 8.2 parts by weight of a synthetically prepared glycine, and the mixture is slowly stirred for about two more hours. A hazy blue solution is observed. The synthetic production method for preparing the glycine is by reacting aldehydes with ammonia and hydrogen cyanide, followed by hydrolysis of the resulting α-aminonitriles. To the hazy blue solution is added 65 parts by weight of a 15 wt % citric acid solution and the mixture is stirred until a clear blue solution is observed. This solution is spray dried resulting in a copper bisglycinate powder having a copper content of about 14 wt % and which melted at about 194° C. Upon being reconstituted in water, the pH of the resulting solution is about 7.5. All of the compositional components used in the preparation should be determined to be non-GMO.

### Example 4

A mixture of 42.93 grams of zinc sulfate, 12 grams of methionine, and 30 grams of glycine are reacted in an aqueous environment for 60 minutes at a temperature of about 65 to 70° C. The glycine and methionine are prepared using synthetic processes. Specifically, the synthetic production method for preparing the glycine and methionine is by reacting aldehydes with ammonia and hydrogen cyanide, followed by hydrolysis of the resulting α-aminonitriles. The reaction of the zinc sulfate, methionine, and glycine produces a zinc amino acid chelate having a ligand component to metal molar ratio of about 2:1, a theoretical average zinc content of about 26.8% by weight, and a glycine to methionine molar-ratio of about 5:2. Due to the presence of the sulfate anion, the actual average zinc weight percentage is about 18.2%. All of the compositional components used in the preparation should be determined to be non-GMO.

### Example 5

Into about 1300 grams of water is dissolved 210.72 grams of a synthetic glycine and 79.86 grams of calcium oxide. The synthetic production method for preparing the glycine is by reacting aldehydes with ammonia and hydrogen cyanide, followed by hydrolysis of the resulting α-aminonitriles. The solution of calcium oxide and glycine is stirred until all of the calcium oxide appeared to be fully dissolved, i.e. about 15 minutes. The resulting reaction forms a calcium bisglycinate chelate or complex solution. Next, to the calcium bisglycinate chelate or complex solution is added 381.55 grams of ferrous sulfate hydrate containing 20% ferrous iron by weight. Again, the solution is constantly stirred while the ferrous sulfate dissolves and a white precipitate of calcium sulfate forms. About 287 grams of a ferrous glycine chelate is formed having a ligand to metal molar ratio of about 2:1. All of the compositional components used in the preparation should be determined to be non-GMO.

### Example 6

About 2252 grams of water is used to dissolve 450.42 grams of fermentation-produced glycine and 168.24 grams of calcium oxide into solution. The resulting reaction formed a calcium trisglycinate chelate or complex solution. Next, 500.18 grams of chromic sulfate hydrate containing 19 wt % chromium is added to the calcium chelate solution. The solution is stirred while the copper sulfate is dissolved and as a white precipitate of calcium sulfate formed. Upon completion of the reaction, about 545 grams of a chromic trisglycinate chelate having a ligand to metal molar ratio of about 3:1 is formed. All of the compositional components used in the preparation should be determined to be non-GMO.

### Example 7

Into about 923 grams of water is dissolved 150.14 grams of synthetic glycine. The synthetic production method for preparing the glycine is by reacting aldehydes with ammonia and hydrogen cyanide, followed by hydrolysis of the resulting α-aminonitriles. Next, 57.25 grams of calcium oxide, which is about 70 wt % calcium, is added. The solution is continually stirred until all of the calcium oxide is dissolved. This takes about 15 minutes. No heat is applied for this particular reaction. The resulting reaction forms a calcium bisglycinate chelate or complex and water, i.e. the hydrogen ions are removed from the glycine and the oxygen is removed from the calcium oxide. Next, 254.18 grams of copper sulfate hydrate containing 25% copper by weight is added to the calcium chelate solution. Again, the solution is constantly stirred while the copper sulfate is dissolved. As the copper sulfate goes into solution, a white precipitate of calcium sulfate is formed. Upon completion of the reaction, about 214 grams of a copper glycine chelate having a ligand to metal molar ratio of 2:1 is formed. All of the compositional components used in the preparation should be determined to be non-GMO.

### Example 8

About 250 grams of fermentation-produced glycine is dissolved into 937.8 grams of water. Once the glycine is significantly dissolved, about 95 grams of calcium oxide is added. The solution is continually stirred for about 15 minutes until all of the calcium is dissolved. The resulting reaction forms a calcium bisglycinate chelate or complex and water. Next, 299.97 grams of zinc sulfate hydrate containing 35% zinc by weight is added to the calcium chelate solution. Upon constant stirring, the zinc sulfate went into solution and a white precipitate of calcium sulfate is formed. About 355 grams of a zinc glycine chelate having a ligand to metal molar ratio of about 2:1 is also formed. All of the compositional components used in the preparation should be determined to be non-GMO.

### Example 9

An open electrolytic cell is constructed consisting of an anode compartment and a cathode compartment divided by a cation permselective membrane. The anode is pure copper metal, providing the metal to form the chelate at the appropriate time. The volume of the anode compartment is approximately 400 cc and the volume of the cathode compartment is about 650 cc. A transformer and rectifier system is utilized to apply a direct current voltage across the cell. The anolyte solution includes a synthetically produced aqueous glycine having a glycine concentration of about 20%, which is circulated continuously throughout the cell compartment and past the anode. The synthetic production method for preparing the glycine is by reacting aldehydes with ammonia and hydrogen cyanide, followed by hydrolysis of the resulting α-aminonitriles. The catholyte solution is a 1 wt % citric acid solution. The initial temperature of the analyte and catholyte solutions is about 40° C. The applied voltage to the transformer is 75 V A.C. The initial voltage across the cell is 5 V D.C. at an amperage of 27 amps. The temperature within each compartment rises quite rapidly and levels off at about 90° C. in the anode compartment and 94° C. in the cathode compartment. The amperage slowly increases to about 34 amps and then remains constant and the voltage across the cell decreases slowly during the entire hour of operation from 5 V D.C. to 2.2 V D.C. Upon cooling to room temperature, a blue precipitate is formed and separates from the anolyte solution. Upon assay, the blue precipitate is shown to be a copper glycine chelate containing 6% copper and having a ligand to copper ration of 2:1. The resulting chelate precipitate is free of any anions. The current flow between the anode and cathode compartments is made possible by the migration of hydrogen ions through the cation permselective membrane. Also, upon cooling it is found that certain of the copper ions had also migrated through the membrane and are loosely plated on the cathode. All of the compositional components used in the preparation should be determined to be non-GMO.

### Example 10

The amino acid chelate prepared in accordance with Example 1 is spray dried and blended with non-GMO fumed silica (about 0.1 wt % to 5 wt % of composition) and non-GMO maltodextrin (about 0.1 wt % to 85 wt % of composition). A free flowing powder having acceptable hydroscopicity is formed.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

1. A non-GMO metal amino acid chelate composition, comprising a metal amino acid chelate including a naturally occurring amino acid chelated to a metal, said amino acid to metal molar ratio being from about 1:1 to 4:1, wherein both the amino acid and a source of the metal used to form the amino acid chelate are non-GMO.

2. A composition as in claim 1, wherein the naturally occurring amino acid is selected from the group consisting of alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and combinations thereof.

3. A composition as in claim 1, wherein the metal is selected from the group consisting of iron, zinc, copper, manganese, calcium, magnesium, chromium, vanadium, selenium, silicon, molybdenum, tin, nickel, boron, cobalt, gold, silver, and combinations thereof.

4. A composition as in claim 1, wherein the metal is a polyvalent metal, and the naturally occurring amino acid to metal molar ratio is from about 1:1 to 3:1.

5. A composition as in claim 1, wherein the metal is ferrous iron and the naturally occurring amino acid is glycine, and wherein the iron to glycine molar ratio is about 2:1.

6. A composition as in claim 1, wherein the metal is copper and the naturally occurring amino acid is glycine, and wherein the copper to glycine molar ratio is about 2:1.

7. A composition as in claim 1, wherein the metal is zinc and the naturally occurring amino acid is glycine, and wherein the zinc to glycine molar ratio is about 2:1.

8. A composition as in claim 1, wherein the metal is manganese and the naturally occurring amino acid is glycine, and wherein the manganese to glycine molar ratio is about 2:1.

9. A composition as in claim 1, wherein the metal is ferric iron and the naturally occurring amino acid is glycine, and wherein the ferric iron to glycine molar ratio is about 3:1.

10. A composition as in claim 1, wherein the metal is chromium and the naturally occurring amino acid is glycine, and wherein the iron to glycine molar ratio is about 3:1.

11. A composition as in claim 1, wherein the metal is magnesium and the naturally occurring amino acid is glycine, and wherein the magnesium to glycine molar ratio is about 1:1.

12. A composition as in claim 1, wherein the metal is calcium and the naturally occurring amino acid is glycine, and wherein the calcium to glycine molar ratio is about 1:1.

13. A composition as in claim 1, wherein the naturally occurring amino acid used to prepare the amino acid chelates is prepared by a method other than protein hydrolysis.

14. A composition as in claim 13, wherein the naturally occurring amino acid used to prepare the amino acid chelates is prepared synthetically.

15. A composition as in claim 13, wherein the naturally occurring amino acid used to prepare the amino acid chelates is prepared by fermentation.

16. A composition as in claim 1, wherein the naturally occurring amino acid used to prepare the amino acid chelates is prepared by protein hydrolysis, and wherein the protein used in the hydrolysis is non-GMO.

17. A non-GMO metal amino acid chelate-containing composition, comprising:
a) a non-GMO metal amino acid chelate including a naturally occurring amino acid chelated to a metal, said amino acid to metal molar ratio being from about 1:1 to 4:1, wherein both the amino acid and a source of the metal used to form the amino acid chelate are non-GMO; and
b) a non-GMO formulation additive,
with the proviso that all components of the non-GMO metal amino acid chelate-containing composition are non-GMO.

18. A composition as in claim 17, wherein the formulation additive is a non-GMO organic acid.

19. A composition as in claim 18, wherein the non-GMO organic acid is selected from the group consisting of citric acid, fumaric acid, succinic acid, tartaric acid, malic acid, lactic acid, gluconic acid, ascorbic acid, pantothenic acid, folic acid, lipoic acid, oxalic acid, maleic acid, formic acid, acetic acid, pyruvic acid, adipic acid, alpha-ketoglutaric acid, and mixtures thereof.

20. A composition as in claim 17, wherein the formulation additive is a non-GMO filler.

21. A composition as in claim 20, wherein the non-GMO filler is selected from the group consisting of grain flours, maltodextrins, vegetable flours or powders, inulin, and combinations thereof.

22. A composition as in claim 17, wherein the formulation additive is a non-GMO flow control agent.

23. A composition as in claim 22, wherein the non-GMO flow control agent is selected from the group consisting of fumed silica, stearic acid, talc, and combinations thereof.

24. A composition as in claim 17, wherein the formulation additive is selected from the group consisting of free amino acids, amino acid salts, and combinations thereof.

25. A composition as in claim 17, wherein the formulation additive is selected from the group consisting of vitamins, coenzymes, cofactors, herbs, herbal extracts, protein powders, and combinations thereof.

26. A composition as in claim 17, wherein the formulation additive is selected from the group consisting of mineral oils, binders, flavoring or taste-free additives, and combinations thereof.

27. A composition as in claim 17, wherein the naturally occurring amino acid is selected from the group consisting of alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and combinations thereof.

28. A composition as in claim 17, wherein the metal is selected from the group consisting of iron, zinc, copper, manganese, calcium, chromium, vanadium, selenium, silicon, molybdenum, tin, nickel, boron, cobalt, gold, silver, and combinations thereof.

29. A composition as in claim 17, wherein the metal is a polyvalent metal, and the naturally occurring amino acid to metal molar ratio is from about 1:1 to 3:1.

30. A composition as in claim 17, wherein the naturally occurring amino acid used to prepare the amino acid chelates is prepared by a method other than protein hydrolysis.

31. A composition as in claim 30, wherein the naturally occurring amino acid used to prepare the amino acid chelates is prepared synthetically.

32. A composition as in claim 30, wherein the naturally occurring amino acid used to prepare the amino acid chelates is prepared by fermentation.

33. A composition as in claim 17, wherein the naturally occurring amino acid used to prepare the amino acid chelates is prepared by protein hydrolysis, and wherein the protein used in the hydrolysis is non-GMO.

34. A method of preparing a non-GMO metal amino acid chelate, comprising:
a) selecting an amino acid source determined to be non-GMO;
b) selecting a metal source determined to be non-GMO; and
c) chelating an amino acid of the amino acid source to a metal of the metal source, thereby forming a non-GMO metal amino acid chelate.

35. A method as in claim 34, wherein during the step of selecting the amino acid source, if a first amino acid source is a GMO, additional amino acid sources are evaluated until a non-GMO amino acid source is ascertained.

36. A method as in claim 34, wherein during the step of selecting the metal source, if a first metal source is a GMO, additional metal sources are evaluated until a non-GMO metal source is ascertained.

37. A method as in claim 34, wherein the naturally occurring amino acid used to prepare the amino acid chelates is prepared by a method other than protein hydrolysis.

38. A method as in claim 37, wherein the naturally occurring amino acid used to prepare the amino acid chelates is prepared synthetically.

39. A composition as in claim 37, wherein the naturally occurring amino acid used to prepare the amino acid chelates is prepared by fermentation.

40. A method as in claim 34, wherein the naturally occurring amino acid used to prepare the amino acid chelates is prepared by protein hydrolysis, and wherein the protein used in the hydrolysis is non-GMO.

41. A method as in claim 34, further comprising selecting an additive determined to be non-GMO, and including the additive as a mixture with the non-GMO metal amino acid chelate.

42. A method as in claim 41, wherein the additive is selected from the group consisting of non-GMO organic acids, non-GMO free amino acids, non-GMO amino acid salts, non-GMO fillers, non-GMO flow control agents, non-GMO lubricants, non-GMO flow agents, non-GMO hydroscopicity minimizing agents, non-GMO pH control agents, non-GMO catalysts, non-GMO vitamins, non-GMO dust control agents, non-GMO binders, non-GMO disintegrating agents, non-GMO flavoring agents, non-GMO taste-reducing agents, non-GMO capsule shells, non-GMO shellacs, non-GMO waxes, non-GMO emulsifiers, non-GMO oils, and combinations thereof.

43. A method of administering a metal amino acid chelate, comprising:
a) formulating a non-GMO metal amino acid chelate by:
i) selecting an amino acid source determined to be non-GMO,
ii) selecting a metal source determined to be non-GMO, and
iii) chelating an amino acid of the amino acid source to a metal of the metal source, thereby forming the non-GMO metal amino acid chelate; and
b) administering the non-GMO metal amino acid chelate to the subject.

44. A method as in claim 43, wherein during the step of selecting the amino acid source, if a first amino acid source is a GMO, additional amino acid sources are evaluated until a non-GMO amino acid source is ascertained.

45. A method as in claim 43, wherein during the step of selecting the metal source, if a first metal source is a GMO, additional metal sources are evaluated until a non-GMO metal source is ascertained.

46. A method as in claim 43, wherein the naturally occurring amino acid used to prepare the amino acid chelates is prepared by a method other than protein hydrolysis.

47. A method as in claim 46, wherein the naturally occurring amino acid used to prepare the amino acid chelates is prepared synthetically.

48. A composition as in claim 46, wherein the naturally occurring amino acid used to prepare the amino acid chelates is prepared by fermentation.

49. A method as in claim 43, wherein the naturally occurring amino acid used to prepare the amino acid chelates is prepared by protein hydrolysis, and wherein the protein used in the hydrolysis is non-GMO.

50. A method as in claim 43, further comprising selecting an additive determined to be non-GMO, and including the additive as a mixture with the non-GMO metal amino acid chelate.

51. A method as in claim 50, wherein the additive is selected from the group consisting of non-GMO organic acids, non-GMO free amino acids, non-GMO amino acid salts, non-GMO fillers, non-GMO flow control agents, non-GMO lubricants, non-GMO flow agents, non-GMO hydroscopicity minimizing agents, non-GMO pH control agents, non-GMO catalysts, non-GMO vitamins, non-GMO dust control agents, non-GMO binders, non-GMO disintegrating agents, non-GMO flavoring agents, non-GMO taste-reducing agents, non-GMO capsule shells, non-GMO shellacs, non-GMO waxes, non-GMO emulsifiers, non-GMO oils, and combinations thereof.
